(19) United States
(12) Patent Application Publication
(10) Pub. No.: US 2008/0255079 A1
(43) Pub. Date: Oct. 16, 2008

(54) Therapeutic Use of Nefopam and Analogues Thereof

(76) Inventors: Michael Harvey Lyne, Essex (GB); Robin Mark Bannister, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/910,549

(22) PCT Filed: Mar. 31, 2006
(86) PCT No.: PCT/GB06/01197

§ 371 Date: Jun. 1, 2008

(30) Foreign Application Priority Data
Apr. 4, 2005 (GB) 0506835.8

(51) Int. Cl. A61K 31/33; A61P 43/00

(52) U.S. Cl. 514/183

(57) ABSTRACT

Nefopam or an analogue thereof is useful in the treatment of a syndrome characterized by chronic pain and fatigue.

### FIELD OF THE INVENTION

This invention relates to a new therapeutic use of nefopam and analogues thereof.

### BACKGROUND OF THE INVENTION

Nefopam, i.e. 5-methyl-1-phenyl-3,4,5,6-tetrahydro-1H-2,5-benzoxazocine hydrochloride, is a centrally acting non-narcotic analgesic not structurally related to other analgesics. Nefopam has been shown to induce antinociception in animal models of pain and in humans. Although the precise mechanism of antinociception is not known, it is thought to involve inhibition of synaptosomal uptake of dopamine, norepinephrine and serotonin.

In vitro and in vivo studies with nefopam enantiomers have shown that (+)-nefopam has more potent analgesic and dopamine, norepinephrine and serotonin uptake inhibitory properties than (−)-nefopam, with the order of potency given as (+)-nefopam>(±)-nefopam>(−)-nefopam (Fasmer et al., 1987; Rosland and Hole, 1990; Mather et al., 2001). Although the study of Mather et al. concludes that there is currently no compelling rationale to justify administering or monitoring individual enantiomers of nefopam, there may be advantages in using the single enantiomers of nefopam for the treatment of pain and emesis. These utilities are disclosed in, inter alia, WO03/105832 and WO03/105833.

Conventional release preparations of nefopam have been commercially available for many years, for use in moderate to severe pain, yet the short elimination half-life of nefopam (four hours) means that it is difficult to maintain analgesic efficacy over the normal dosing period (three times daily). Dose escalation of nefopam brings about an increase in the frequency of adverse drug reactions associated with the analgesic and adverse effects on pulse and blood pressure have been observed following parenteral delivery of therapeutic doses of nefopam (Heel et al., 1980). The chronotropic and ionotropic effects of nefopam on the heart are not present when nefopam is administered orally (Bhatt et al., 1981).

WO2004/056788, WO2005/103019 and US2006/0019940 disclose analogues of nefopam.

Fibromyalgia is a chronic condition characterised by fatigue and widespread pain in muscles, ligaments and tendons. The widespread musculo-skeletal pain often presents with a number of co-morbidities including fatigue, sleep disturbance, anxiety and depression. Affected people are predominantly women. This condition (also known, usually in the past, as fibrositis, chronic muscle pain syndrome, psychogenic rheumatism or tension myalgia) is poorly understood, and it remains poorly treated. Related syndromes include chronic fatigue syndrome, complex regional pain syndrome, irritable bowel syndrome, myofacial pain and atypical chest pain. Anxiolytics/antidepressants have shown some success in the clinic in alleviating symptoms of fibromyalgia (Sayar K. et al., 2003—Ann Pharmacother. 37(11):1561-1565; Pagano T. et al., 2004—Sao Paulo Med. J. 122(6):252-258).

### SUMMARY OF THE INVENTION

The present invention is based on the realisation that nefopam may have utility in the treatment of syndromes characterised by chronic pain and fatigue, especially when given in a controlled-release formulation. These syndromes include, but are not limited to, fibromyalgia, chronic fatigue syndrome, complex regional pain syndrome, irritable bowel syndrome, myofacial pain and atypical chest pain. Controlled release may extend the analgesic effect and reduce the occurrence of side-effects associated with plasma peak concentrations of an immediate release product.

As used herein, “nefopam” refers to a compound of formula I

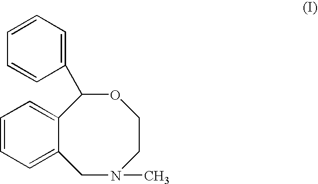

and salts, e.g. the hydrochloride, metabolites and prodrugs thereof, as well as the (+) and (−) enantiomers which are as far as possible optically pure. (+)-Nefopam may be preferred, e.g. for reduced side-effects that may be caused by interaction.

An analogue of nefopam may be used. Such compounds are described in WO2004/056788, WO2005/103019 and US2006/0019940, the content of each of which is incorporated herein by reference.

### DESCRIPTION OF PREFERRED EMBODIMENTS

According to the invention, the active compound is used to treat patients exhibiting syndromes characterised by chronic pain and fatigue. These syndromes include, but are not limited to, fibromyalgia, chronic fatigue syndrome, complex regional pain syndrome, irritable bowel syndrome, myofacial pain and atypical chest pain. Any suitable route of administration can be used. For example, any of oral, topical, ocular, rectal, vaginal, inhalation and intranasal delivery routes may be suitable. The dose of the active agent will depend on the nature and degree of the condition, the age and condition of the patient, and other factors known to those skilled in the art. A typical dosage is 10-100 mg given one to three times per day.

If controlled release of the active agent is required, a suitable formulation of any type known to those skilled in the art may be used. Modified release can be afforded by either dissolution or diffusion controlled monolithic devices, beaded encapsulated systems, osmotically controlled systems, and modified film coating systems incorporating suitable polymeric and non-polymeric hydrophilic and hydrophobic materials. Suitable controlled-release formulations include hydrophilic materials comprising, but not limited to, acrylic or methacrylic polymers or copolymers, alkylvinyl polymers, celluloses, hydroxyalkyl celluloses, carboxyalkyl celluloses, polysaccharides, alginates, pectins, starches and derivatives, natural and synthetic gums, polycarbophil and chitosans. Suitable hydrophobic materials comprise, but are not limited to, hydrophobic polymers, waxes, fats, long-chained fatty acids, their corresponding esters, their corresponding ethers, and their mixtures.

It will often be advantageous to use nefopam in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Currently, there is no single pre-clinical model considered sufficiently representative of fibromyalgia syndrome. However, in view of its aetiology, efficacy in models which represent persistent painful states (e.g. formalin-induced hyperalgesia) or demonstrated antidepressant/anxiolytic activity may be relevant to efficacy in fibromyalgia. Compounds which show activity in both the formalin test and in the behavioural tests might be expected to have utility in treating the symptoms of fibromyalgia.

The following studies provide evidence on which the present invention is based.

### Formalin-Induced Hyperalgesia in Mouse

Nefopam and (+)-nefopam have been evaluated in the formalin-induced paw licking model in mice. The two phases of the mouse formalin test have been shown to have different nociceptive mechanisms (Hunskaar S. & Hole K., 1987—Pain 30(1):103-114). It is suggested that the early phase is due to a direct effect on nociceptors and resembles acute nociceptive pain. The late phase seems to be an inflammatory response and is a recognized model of persistent pain. This test may, therefore, be a useful indicator of analgesic efficacy in fibromyalgia. Compounds were evaluated for both an early stage response and a late stage response and compared against morphine as control.

Inflammation was induced by subplantar injection of a 5% formalin solution (0.02 ml) into the mouse right hindpaw (20-25 g male Rj: NMRI). Hindpaw licking time was continuously recorded in a blinded fashion between 0 to 5 minutes (early phase) and between 20 to 30 minutes (late phase) after formalin injection (Hunskaar etal., 1985—J. Neurosci. Methods; 14:69-76).

The test substances and vehicle were orally administered 60 min before formalin injection. Results are shown in Table 1.

TABLE 1

| | | | |
|---|---|---|---|
| Nociception | Morphine | Nefopam | (+)-Nefopam |

Early Phase:

| | | | |
|---|---|---|---|
| 30 mg/kg po | nt | −28% | −40% |
| 60 mg/kg po | nt | −64%* | −59%* |
| 80 mg/kg po | −98%* | nt | nt |
| 100 mg/kg po | nt | −85%* | −86%* |

Late Phase:

| | | | |
|---|---|---|---|
| 30 mg/kg po | nt | −41% | −31% |
| 60 mg/kg po | nt | −63%* | −69% |
| 80 mg/kg po | −98%* | nt | nt |
| 100 mg/kg po | nt | −89%* | −90%* |

nt = not tested;
*Denotes statistical significance achieved

Both nefopam and (+)-nefopam dose-dependently decreased paw licking time when compared to vehicle control. In the first phase (representative of acute nociceptive pain), nefopam and (+)-nefopam showed a significant reduction in licking behaviour compared to vehicle control at both 60 and 100 mg/kg. In the second phase (representative of persistent pain states), nefopam and (+)-nefopam showed a significant reduction in licking behaviour at 100 mg/kg. The data demonstrate that both nefopam and (+)-nefopam have significant analgesic efficacy in acute nociceptive and persistent pain states.

### Behavioural Despair Test in Mouse

Nefopam and (+)-nefopam have been evaluated in the Behavioural Despair Test, a model which detects antidepressant activity. This test was conducted according to the method of Porsolt et al., (1977—Arch. Int. Pharmacodyn., 229:327-336), in which mice are forced to swim in a situation from which they cannot escape rapidly become immobile. Antidepressants decrease the duration of immobility.

Mice (20-27 g male Rj: NMRI) were individually placed in a cylinder (height=24 cm, diameter=13 cm) containing 10 cm water (22° C.) from which they cannot escape. The mice were placed in the water for 6 minutes and the duration of immobility during the last 4 minutes was measured. All compounds were administered i.p. 30 minutes before the test, and compared with a vehicle control group. Imipramine (32 mg/kg i.p.), administered under the same experimental conditions, was used as reference substance. Results are shown in Table 2.

TABLE 2

| Duration of Immobility | | | |
|---|---|---|---|
| (% change from ctrl) | Nefopam | (+)-nefopam | Imipramine |
| 20 mg/kg ip | −42 | −38 | nt |
| 32 mg/kg ip | nt | nt | −80 * |
| 40 mg/kg ip | −85 * | −92 * | nt |
| 60 mg/kg ip | −100 * | −100 * | nt |

nt = not tested;
* Denotes statistical significance achieved

The data demonstrate that both nefopam and (+)-nefopam have significant anti-depressant activity.

### Marble Burying Test in Mouse

Nefopam and (+)-nefopam have been evaluated in the Marble Burying Test, a model which detects anxiolytic/tranquillizing activity. The method follows that described by Broekkamp et al. (1986—Eur. J. Pharmacol., 126, 223-229). Mice exposed to novel object (marbles) will bury them in the sawdust floor covering. Anxiolytics decrease the number of marbles buried at non-sedative doses.

Mice are individually placed in transparent plastic cages (33×21×18 cm) with 5 cm of sawdust on the floor, and 25 marbles grouped in the centre of the cage. The cage is covered with an inverted plastic cage. Each test cage, together with the marbles, is impregnated with mouse odor beforehand, by leaving 10 mice in the cage for 15 minutes. These mice then play no further role in the experiment. The number of marbles covered by sawdust (⅔ or more) is counted at the end of a 30 minute test.

All compounds were administered i.p. 30 minutes before the test, and compared with a vehicle control group. Clobazam (8 mg/kg i.p.), administered under the same experimental conditions, was used as reference substance. Results are given in Table 3.

TABLE 3

| Number of marbles buried (% | | | |
|---|---|---|---|
| change from ctrl) | Nefopam | (+)-nefopam | Clobazam |
| 10 mg/kg ip | −66 * | −56 * | nt |
| 20 mg/kg ip | −98 * | −99 * | Nt |
| 32 mg/kg ip | Nt | nt | −75 * |
| 40 mg/kg ip | −100 * | −100 * | Nt |

nt = not tested;
* Denotes statistical significance achieved

The data demonstrate that both nefopam and (+)-nefopam have significant antidepressant and anxiolytic activity.

### Parallel Group Study

The utility is further demonstrated in a Phase Ha multi-centre, randomised, double-blind, placebo-controlled, parallel group study. A total of 100 subjects are randomised to receive nefopam as racemate, either enantiomer or placebo 3 times daily for 28 days.

The study consists of three periods:

Washout: 3 to 30 days prior to randomisation, subjects undergo a screening visit to determine eligibility (Visit 1). At this visit, eligible subjects are advised to discontinue central nervous system active therapies, including antidepressants, sedative-hypnotic agents, muscle relaxants and centrally-acting analgesics.

Treatment: Eligible subjects are randomised at Baseline (Visit 2) to receive Nefopam as racemate, either enantiomer or placebo in a 1:1 ratio. Subjects take a single oral capsule 3 times daily for 28 days. They return to the unit at weeks 1 (Visit 3), 2 (Visit 4), 3 (Visit 5) and 4 (Visit 6).

Follow-up: Subjects return for an End of Study visit (Visit 7) 2 weeks after the end of treatment.

During the Treatment period, patients complete the Fibromyalgia Impact Questionnaire (FIQ), Short Form-McGill Pain Questionnaire (SF-MPQ), Hospital Anxiety and Depression Scale (HADS) and Fibromyalgia Health Assessment Questionnaire (FHAQ) to assess any changes in symptoms during treatment. In addition, any changes in the extent of musculoskeletal pain are measured. The primary endpoint for the study is the FIQ total score after 4 weeks treatment. Secondary endpoints are:
(i) FIQ total score at weeks 1, 2, 3, at end of study and overall.
(ii) FIQ sub-scales at weeks 1, 2, 3, 4, at end of study and overall.
(iii) SF-MPQ sub-scales at weeks 1, 2, 3, 4, at end of study and overall.
(iv) Tender point assessment (from the ACR 1990 criteria) scores at weeks 2, 4, at end of study and overall.
(v) HADS sub-scales at weeks 2, 4 weeks and overall.
(vi) FHAQ total score at weeks 1, 2, 3, 4, at end of study and overall.

1. A method for the treatment of a syndrome characterised by chronic pain and fatigue, wherein the method comprises administering, to a patient in need of such treatment, a compound that is nefopam or is of any of the formulae 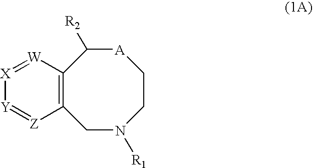
wherein R₁ is H, C₁-C₆ alkyl optionally substituted with F or C₃-C₆ cycloalkyl or C₂-C₄ alkenyl;
A is O, CH₂ or S(O)ₙ where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O)ₙ where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆ alkyl, C₂-C₆ alkenyl, C₂-C₆ alkynyl, C₃-C₆ cycloalkyl, aryl or heteroaryl; and
R₅ is H, C₁-C₆ alkyl, C₂-C₆ alkenyl, C₂-C₆ alkynyl, C₃-C₆ cycloalkyl, aryl or heteroaryl and is the same as or different to another R₅;
or a pharmaceutically acceptable salt thereof; 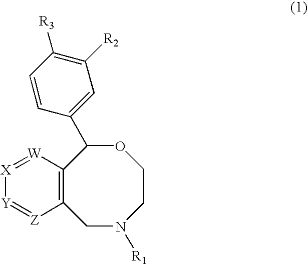
wherein
R₁ is H, C₁-C₆ alkyl, optionally substituted with F or C₃-C₆ cycloalkyl or C₂-C₆ alkenyl;
R₂ and R₃ are the same or different and are H, a halogen, CN, CF₃, C₁-C₆ alkyl or OR₁, or R₂ and R₃ form a five or six membered ring which may be carbocyclic, heterocyclic (containing 1-2 heteroatoms taken from O, N and S), aromatic or heteroaromatic (containing 1-2 heteroatoms taken from O and N);
one of W, X, Y and Z is N, or CR₄ and the others are each CH;
R₄ is a halogen atom, CF₃, CN, OR₇, SO₂N(R₆)₂, COR₆, CO₂R₆, CON(R₆)₂, NR₁COR₅, NR₁SO₂R₅, NR₁CO₂R₅, NR₁CON(R₆)₂, OC₁-C₆ alkyl optionally substituted with R₄, C₁-C₆ alkyl optionally substituted with R₄, C₃-C₆ cycloalkyl optionally substituted with R₄, C₂-C₆ alkenyl optionally substituted with R₄, C₂-C₆ alkynyl optionally substituted with R₄, aryl optionally substituted with R₄, or a five or six membered aromatic heterocycle containing 1-4 heteroatoms selected from N and O, linked either through carbon or nitrogen;
R₅ is C₁-C₆ alkyl, C₂-C₆ alkenyl, C₂-C₆ alkynyl, C₃-C₆ cycloalkyl, aryl or heteroaryl;
each R₆ (which may be the same or different) is H, C₁-C₆ alkyl, C₂-C₆ alkenyl, C₂-C₆ alkynyl, C₃-C₆ cycloalkyl, aryl or heteroaryl; and
R₇ is aryl or heteroaryl;
or a pharmaceutically acceptable salt thereof; 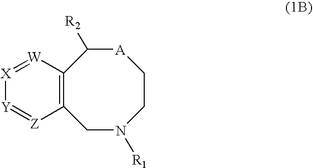
wherein R₁ is H, C₁-C₆ alkyl optionally substituted with F or C₃-C₆ cycloalkyl or C₂-C₄ alkenyl;
A is O, CH₂ or S(O)ₙ where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O)ₙ where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂R₄; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆ alkyl, C₂-C₆ alkenyl, C₂-C₆ alkynyl, C₃-C₆ cycloalkyl, aryl or heteroaryl; and
R₅ is H, C₁-C₆ alkyl, C₂-C₆ alkenyl, C₂-C₆ alkynyl, C₃-C₆ cycloalkyl, aryl or heteroaryl and is the same as or different to another R₅;
or a pharmaceutically acceptable salt thereof; 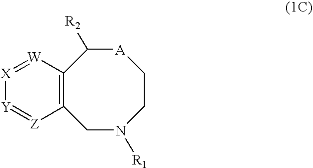
wherein R₁ is H, C₁-C₆ alkyl optionally substituted with F or C₃-C₆ cycloalkyl or C₂-C₄ alkenyl;
A is O, CH₂ or S(O)ₙ where n is 0-2;
one of W, X, Y and Z is N, CH or CR₃ and the others are CH;
R₂ is C₅-C₆ heteroaryl, C₅-C₁₀ cycloalkyl or cycloalkenyl optionally containing one or more heteroatoms selected from O, N and S(O)ₙ where n is 0-2, and optionally substituted with R₃; or a phenyl group optionally substituted in one or more positions with one or more substituents independently selected from halogen, CN, CF₃, C₁-C₆ alkyl and OR₁, or the phenyl group is fused to a five or six membered ring which may be carbocyclic, heterocyclic (containing 1-2 heteroatoms selected from O, N and S), aromatic or heteroaromatic (containing 1-2 heteroatoms selected from O and N);
R₃ is selected from halogen; CF₃; CN; OR₅; SO₂N(R₅)₂; COR₅; CO₂R₅; CON(R₅)₂; NR₁COR₄; NR₁SO₂ ; NR₁CO₂R₄; NR₁CON(R₅)₂; OC₁-C₆ alkyl substituted with R₃; C₁-C₆ alkyl optionally substituted with unsubstituted R₃; C₃-C₆ cycloalkyl optionally substituted with unsubstituted R₃; C₂-C₆ alkenyl optionally substituted with unsubstituted R₃; C₂-C₆ alkynyl optionally substituted with unsubstituted R₃; aryl optionally substituted with unsubstituted R₃; and five or six membered aromatic heterocycles containing 1-4 heteroatoms selected from N and O;
R₄ is C₁-C₆ alkyl, C₂-C₆ alkenyl, C₂-C₆ alkynyl, C₃-C₆ cycloalkyl, aryl or heteroaryl; and
R₅ is H, C₁-C₆ alkyl, C₂-C₆ alkenyl, C₂-C₆ alkynyl, C₃-C₆ cycloalkyl, aryl or heteroaryl and is the same as or different to another R₅;
or a pharmaceutically acceptable salt thereof; or 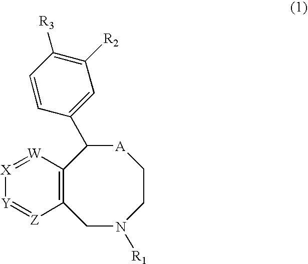
wherein:
R₁ is H, C₁-C₆ alkyl, optionally substituted with F or C₃-C₆ cycloalkyl or C₂-C₄ alkenyl;
R₂ and R₃ are the same or different and are each H, halogen, CN, CF₃, C₁-C₆ alkyl or OR₁, or R₂ and R₃ may form a five or six membered ring which may be carbocyclic, heterocyclic (containing 1-2 heteroatoms taken from O, N and S), aromatic or heteroaromatic (containing 1-2 heteroatoms taken from O and N); and
one of W, X, Y and Z is N, CH or CR₄ and the others are CH;
R₄ is halogen; CF₃; CN; OR₇; SO₂N(R₆)₂ (where each R₆ is the same or different); COR₆; CO₂R₆; CON(R₆)₂ (where R₆ is the same or different); NR₁COR₅; NR₁SO₂R₅; NR₁CO₂R₅; NR₁CON(R₆)₂ (where each R₆ is the same or different), OC₁-C₆ alkyl substituted with unsubstituted R₄, C₁-C₆ alkyl optionally substituted with unsubstituted R₄, C₃-C₆ cycloalkyl optionally substituted with unsubstituted R₄, C₂-C₆ alkenyl optionally substituted with unsubstituted R₄, C₂-C₆ alkynyl optionally substituted with unsubstituted R₄ and aryl optionally substituted with unsubstituted R₄, or R₄ is a five or six membered aromatic heterocycle containing 1-4 heteroatoms taken from N and O;
R₅ is C₁-C₆alkyl, C₂-C₆alkenyl, C₂-C₆ alkynyl, C₃-C₆cycloalkyl, aryl or heteroaryl;
R₆ can be H, C₁-C₆alkyl, C₂-C₆alkenyl, C₂-C₆ alkynyl, C₃-C₆cycloalkyl, aryl or heteroaryl; and
R₇ is aryl or heteroaryl;
or a pharmaceutically acceptable salt thereof.

2. The method, according to claim 1, wherein the syndrome is fibromyalgia.

3. The method, according to claim 1, wherein the syndrome is chronic fatigue syndrome, complex regional pain syndrome, irritable bowel syndrome, myofacial pain or atypical chest pain.

4. The method, according to claim 1, wherein the medicament provides controlled or delayed release of the nefopam.

5. The method, according to claim 1, wherein the nefopam is in the form of the racemate.

6. The method, according to claim 1, wherein the nefopam is in the form of the (+)-enantiomer, substantially free of (−)-nefopam.
